(19) United States
(12) Patent Application Publication
(10) Pub. No.: US 2026/0240592 A1
(43) Pub. Date: Aug. 20, 2026

(54) LEAFLET LACERATION DEVICE

(71) Applicant: BOSTON SCIENTIFIC SCIMED, INC., MAPLE GROVE, MN (US)

(72) Inventors: James M. Anderson, Corcoran, MN (US); Phil Litecky, Forest Lake, MN (US); Eric Michael Petersen, Maple Grove, MN (US); Lauren Koon, Etobicoke (CA)

(73) Assignee: BOSTON SCIENTIFIC SCIMED, INC., MAPLE GROVE, MN (US)

(21) Appl. No.: 19/541,804

(22) Filed: Feb. 17, 2026

Related U.S. Application Data
(60) Provisional application No. 63/759,357, filed on Feb. 17, 2025.

(51) Int. Cl. A61B 18/14; A61B 18/00

(52) U.S. Cl. CPC A61B 18/1492; A61B 2018/00077; A61B 2018/00083; A61B 2018/00244; A61B 2018/00369; A61B 2018/00595; A61B 2018/00601

(57) ABSTRACT

A system for lacerating a tissue includes a catheter including one or more lacerators, where the one or more lacerators are configured to lacerate the tissue at a first exposure window and a second exposure window. The system also includes one or more aligners, where the one or more aligners are deployable and configured to, when deployed, promote contact between the one or more lacerators and the tissue at the first exposure window and/or the second exposure window.

### CROSS REFERENCE TO RELATED APPLICATIONS

This application claims the benefit of priority of U.S. Provisional Application No. 63/759,357 filed Feb. 17, 2025, the entire disclosure of which is hereby incorporated by reference.

### TECHNICAL FIELD

The present disclosure relates generally to medical devices. More particularly, the present disclosure pertains to medical devices for lacerating cardiac valve leaflets.

### BACKGROUND

A wide variety of intracorporeal medical devices have been developed for medical use, and more specifically for intravascular use. Some of these devices include devices for lacerating cardiac valve leaflets. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

The disclosure is directed to design, material, manufacturing method, and use alternatives for lacerating cardiac valve leaflets. An example may be found in a medical device for lacerating valve leaflets. The medical device includes an elongate shaft that extends proximally from a distal region and an inflatable balloon that is secured to the distal region. The inflatable balloon has an outer surface and includes a deflated configuration and an inflated configuration. A tubular member extends relative to the elongate shaft and along the outer surface of the inflatable balloon. The tubular member includes a window faced away from the inflatable balloon. An electrocautery electrode is slidingly disposed within the tubular member and is translatable between a position in which the electrocautery electrode is axially spaced apart from the window and a position in which the electrocautery electrode is aligned with the window and is positioned to lacerate a valve leaflet.

Alternatively or additionally, the electrocautery electrode may include an uninsulated portion of an insulated conductive member that is slidingly disposed within the tubular member.

Alternatively or additionally, the uninsulated portion of the insulated conductive member may have a larger diameter than portions of the insulated conductive member on one or both sides of the uninsulated portion.

Alternatively or additionally, the larger diameter uninsulated portion may urge the window open as the larger diameter uninsulated portion approaches the window.

Alternatively or additionally, the insulated conductive member may be adapted to position the electrocautery electrode proximal of the window prior to translating the insulated conductive member distally in order to align the electrocautery electrode with the window.

Alternatively or additionally, the insulated conductive member may be adapted to position the electrocautery electrode distal of the window prior to translating the insulated conductive member proximally in order to align the electrocautery electrode with the window.

Alternatively or additionally, the window may include an opening in the tubular member.

Alternatively or additionally, the window may include an elongate slot formed in the tubular member.

Alternatively or additionally, the tubular member may be adapted such that inflating the inflatable balloon urges the elongate slot open.

Alternatively or additionally, the window may include a preferential tear region formed in the tubular member.

Alternatively or additionally, the preferential tear region may include a perforation formed in the tubular member.

Alternatively or additionally, the valve leaflets may include native valve leaflets.

Another example may be found in a medical device for lacerating valve leaflets. The medical device includes an elongate shaft that extends proximally from a distal region and an expandable element that is secured to the distal region. The expandable element is movable between a compressed configuration and an expanded configuration. A tubular member extends relative to the elongate shaft and exterior to the expandable element. The tubular member includes a side wall and an opening within the side wall. An electrode is slidingly disposed within the tubular member and is translatable between a position in which the electrode is spaced apart from the opening and a position in which the electrode is aligned with the opening.

Alternatively or additionally, the electrode may include an uninsulated portion of an insulated conductive member slidingly disposed within the tubular member.

Alternatively or additionally, the insulated conductive member may be adapted to position the electrode proximal of the opening prior to translating the insulated conductive member distally in order to align the electrode with the window.

Alternatively or additionally, the insulated conductive member may be adapted to position the electrode distal of the opening prior to translating the insulated conductive member proximally in order to align the electrode with the window.

Alternatively or additionally, the valve leaflets may include native valve leaflets.

Another example may be found in a medical device for lacerating valve leaflets. The medical device includes an elongate shaft that extends proximally from a distal region and an inflatable balloon that is secured to the distal region. The inflatable balloon has an outer surface and includes a deflated configuration and an inflated configuration. A tubular member extends relative to the elongate shaft and along the outer surface of the inflatable balloon. The tubular member includes a window faced away from the inflatable balloon. An insulated conductive member including an uninsulated portion thereof forming an electrode is slidingly disposed within the tubular member.

Alternatively or additionally, the uninsulated portion of the insulated conductive member may have a larger diameter than portions of the insulated conductive member on one or both sides of the uninsulated portion.

Alternatively or additionally, the larger diameter uninsulated portion may urge the window open as the larger diameter uninsulated portion approaches the window.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a partial cutaway view showing an aortic heart valve positioned within a native valve annulus of a heart;

FIG. 2 is a schematic view of an illustrative medical device for lacerating valve leaflets;

FIG. 3 is a schematic view of an illustrative medical device for lacerating valve leaflets;

FIG. 4 is a schematic view of a portion of an illustrative medical device for lacerating valve leaflets, showing an electrocautery electrode axially spaced from a window;

FIG. 4A is a cross-sectional view taken along the line 4A-4A of FIG. 4;

FIG. 4B is a cross-sectional view taken along the line 4B-4B of FIG. 4;

FIG. 5 is a schematic view of a portion of an illustrative medical device for lacerating valve leaflets, showing the electrocautery electrode aligned with the window;

FIG. 6 is a schematic view of a portion of an illustrative medical device for lacerating valve leaflets, showing the electrocautery electrode aligned with a window including a preferential tear line;

FIG. 7 is a schematic cross-sectional view taken along the line 7-7 of FIG. 6;

FIG. 8 is a schematic cross-sectional view taken along the line 8-8 of FIG. 6;

FIG. 9 is a schematic cross-sectional view of an illustrative medical device including an inflatable balloon, with the inflatable balloon inflated to a first pressure; and

FIG. 10 is a schematic cross-sectional view of an illustrative medical device including an inflatable balloon, with the inflatable balloon inflated to a second pressure that is greater than the first pressure shown in FIG. 9.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

A number of patients receive artificial heart valves for a variety of reasons including valve malfunction due to calcium accumulation. When an artificial heart valve is implanted, the artificial heart valve may have an expandable frame that presses the native valve leaflets away from the native position of the native valve leaflets. In some instances, the native valve is the aortic valve, and the artificial heart valve is an artificial aortic valve. In some instances, it is possible for one or more of the native valve leaflets, when pressed to the side, to at least partially or even completely block an ostium of one of the coronary arteries. Not only does this present possible health concerns for the patient, particularly if an ostium is completely blocked, but even when an ostium is only partially blocked and thus still permits blood flow, this may present difficulties in subsequently being able to perform balloon angioplasty, or place a stent, in one of the coronary arteries. In some instances, it may be beneficial to slice or lacerate with opportunity to remove or excise one or more of the native valve leaflets prior to implantation of the artificial heart valve so that when the native valve leaflets are pressed to the side by the expandable frame of the artificial heart valve, the native valve leaflets do not block an ostium of any of the coronary arteries.

In some instances, a patient may already have an implanted artificial heart valve such as an artificial aortic valve. The artificial valve leaflets forming part of the already implanted artificial heart valve can be just as problematic with respect to potentially blocking a cardiac artery ostium when displaced to the side when a second artificial heart valve is implanted in place of the first artificial heart valve. The artificial valve leaflets forming part of the artificial heart valve may, for example, be made from porcine or bovine pericardium, or may be polymeric. In some instances, artificial valve leaflets may be made of polymers such as Dacron or Gore-Tex. As discussed here, reference to a valve leaflet may refer to either a native valve leaflet or an artificial valve leaflet.

In some instances, a medical device is adapted for lacerating valve leaflets. The medical device includes an elongate shaft that extends proximally from a distal region and an inflatable balloon that is secured to the distal region. The inflatable balloon has an outer surface and includes a deflated configuration and an inflated configuration. A tubular member extends relative to the elongate shaft and along the outer surface of the inflatable balloon. The tubular member includes a window that is faced away from the inflatable balloon. An electrocautery electrode is slidingly disposed within the tubular member and is translatable between a position in which the electrocautery electrode is axially spaced apart from the window and a position in which the electrocautery electrode is aligned with the window and is positioned to lacerate a valve leaflet.

In some cases, the electrocautery electrode may include an uninsulated portion of an insulated conductive member slidingly disposed within the tubular member. In some cases, the uninsulated portion of the insulated conductive member may have a larger diameter than portions of the insulated conductive member on one or both sides of the uninsulated portion. The larger diameter uninsulated portion may urge the window open as the larger diameter uninsulated portion approaches the window, for example. In some cases, the insulated conductive member may be adapted to position the electrocautery electrode proximal of the window prior to translating the insulated conductive member distally in order to align the electrocautery electrode with the window. In some cases, the insulated conductive member may be adapted to position the electrocautery electrode distal of the window prior to translating the insulated conductive member proximally in order to align the electrocautery electrode with the window.

In some cases, the window may include an opening in the tubular member. As an example, the window may include an elongate slot formed in the tubular member. In some cases, the tubular member may be adapted such that inflating the inflatable balloon urges the elongate slot open. In some cases, the window may include a preferential tear region formed in the tubular member. As an example, the preferential tear region may include a perforation formed in the tubular member. In some cases, the valve leaflets may include native valve leaflets.

In some instances, a medical device is adapted for lacerating valve leaflets. The medical device includes an elongate shaft that extends proximally from a distal region and an expandable element that is secured to the distal region. The expandable element is movable between a compressed configuration and an expanded configuration. A tubular member extends relative to the elongate shaft and exterior to the expandable element. The tubular member includes a side wall and an opening within the side wall. An electrode is slidingly disposed within the tubular member and is translatable between a position in which the electrode is spaced apart from the opening and a position in which the electrode is aligned with the opening.

In some cases, the electrode may include an uninsulated portion of an insulated conductive member slidingly disposed within the tubular member. In some cases, the insulated conductive member may be adapted to position the electrode proximal of the opening prior to translating the insulated conductive member distally in order to align the electrode with the window. In some cases, the insulated conductive member may be adapted to position the electrode distal of the opening prior to translating the insulated conductive member proximally in order to align the electrode with the window. In some cases, the valve leaflets may include native valve leaflets.

In some instances, a medical device is adapted for lacerating valve leaflets. The medical device includes an elongate shaft that extends proximally from a distal region and an inflatable balloon that is secured to the distal region. The inflatable balloon has an outer surface and includes a deflated configuration and an inflated configuration. A tubular member extends relative to the elongate shaft and along the outer surface of the inflatable balloon. The tubular member includes a window faced away from the inflatable balloon. An insulated conductive member includes an uninsulated portion thereof that forms an electrode. The insulated conductive member is slidingly disposed within the tubular member.

In some cases, the uninsulated portion of the insulated conductive member may have a larger diameter than portions of the insulated conductive member on one or both sides of the uninsulated portion. In some cases, the larger diameter uninsulated portion may urge the window open as the larger diameter uninsulated portion approaches the window.

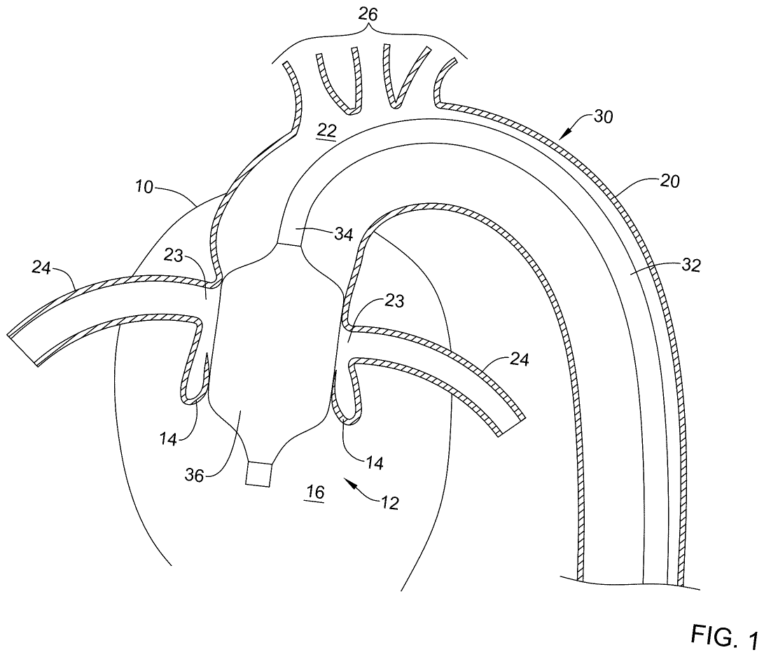

FIG. 1 is a schematic partial cut-away view of a portion of a patient's heart 10 including an aortic valve 12 having native valve leaflets 14 disposed within and/or extending from a native valve annulus, a left ventricle 16, and certain connected vasculature, such as an aorta 20 connected to the aortic valve 12 of the patient's heart 10 by an aortic arch 22 and an ascending aorta, the coronary ostia 23 of the coronary arteries 24, which extend from the aortic sinuses and/or the ascending aorta, and other large arteries 26 (e.g., subclavian and/or carotid arteries, etc.) that extend from the aortic arch 22 to important internal organs. While the aortic valve 12 includes a total of three native valve leaflets 14, only two are visible in the illustrated cutaway view. For the purpose of this disclosure, the discussion herein is directed toward treating the aortic valve 12 and will be so described in the interest of brevity. This, however, is not intended to be limiting as the skilled person will recognize that the following discussion may also apply to other heart valves, vessels, and/or treatment locations within a patient with no or minimal changes to the structure and/or scope of the disclosure.

As shown in FIG. 1, a medical device 30 has been advanced through the aorta 20 and through the aortic arch 22 to a position in which the medical device 30 extends through the native valve leaflets 14. The medical device 30 includes an elongate shaft 32 that extends proximally from a distal region 34. An expandable element 36 is secured to the distal region 34. As shown, the medical device 30 is a balloon catheter, and the expandable element 36 is shown as being an inflatable balloon. In some cases, as will be discussed, the expandable element 36 may not be an inflatable balloon, and may instead include an expandable framework. Regardless, the expandable element 36 may be considered as being movable between a collapsed configuration (a deflated configuration in the case of an inflatable balloon) and an expanded configuration (an inflated configuration in the case of an inflatable balloon), as shown for example in FIG. 1. While the medical device 30 is shown compressing the native valve leaflets 14, it will be appreciated that the medical device 30 may also be used to compress and/or lacerate artificial valve leaflets in a previously implanted replacement heart valve before implanting a new replacement heart valve within the previously implanted replacement heart valve. Some non-limiting examples of a replacement heart valves with which the medical device 30 may be utilized include the ACURATE NEO2™, the ACURATE PRIME™, and/or family members thereof from Boston Scientific of Marlborough, MA, USA.

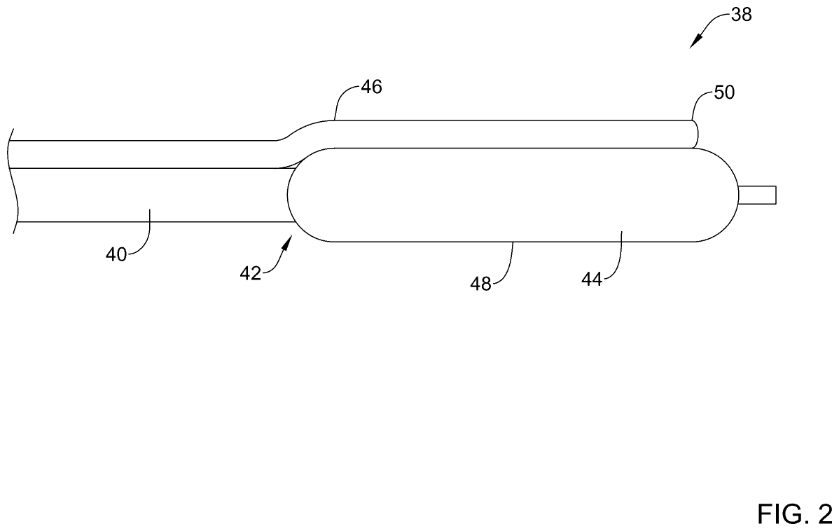

FIG. 2 is a schematic view of an illustrative medical device 38 that may be considered as being an example of the medical device 30 shown in FIG. 1. The medical device 38 may be adapted to lacerate valve leaflets including native valve leaflets 14 as well as being able to lacerate artificial valve leaflets. The medical device 38 includes an elongate shaft 40 that extends proximally from a distal region 42. An inflatable balloon 44 is coupled to the distal region 42 of the elongate shaft 40. The inflatable balloon 44 includes a deflated configuration and an inflated configuration. FIG. 2 shows the inflatable balloon 44 in its inflated configuration. The medical device 38 includes a tubular member 46 that extends relative to the elongate shaft 40 and along an outer surface 48 of the inflatable balloon 44. As will be discussed, the tubular member 46 includes one or more lumens that allows a conductive member forming an electrocautery electrode to be advanced and/or withdrawn within the tubular member 46. In FIG. 2, the tubular member 46 remains exterior to the elongate shaft 40 and includes a distal end 50 that terminates on the outer surface 48 of the inflatable balloon 44. In some cases, the tubular member 46 may be adhesively secured in place relative to the elongate shaft 40 and to the outer surface 48 of the inflatable balloon 44.

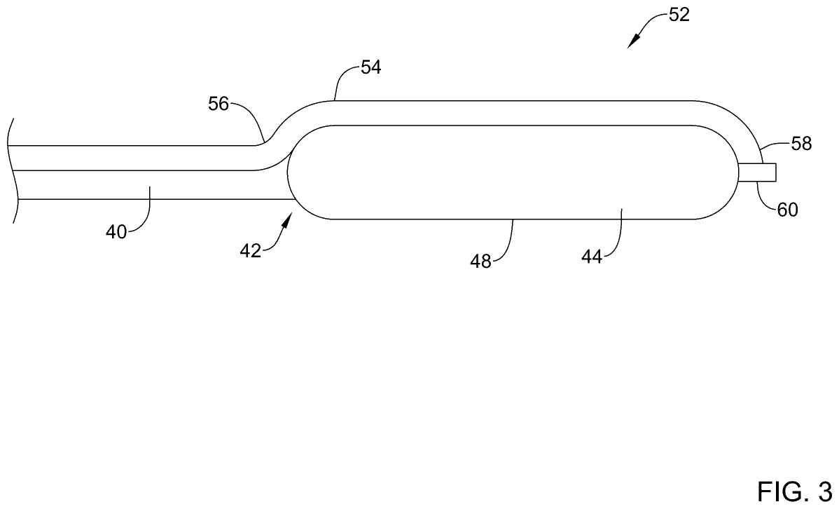

FIG. 3 is a schematic view of an illustrative medical device 52 that may be considered as being an example of the medical device 30 shown in FIG. 1. The medical device 52 may be adapted to lacerate valve leaflets including native valve leaflets 14 as well as being able to lacerate artificial valve leaflets. The medical device 52 includes an elongate shaft 40 that extends proximally from a distal region 42. An inflatable balloon 44 is coupled to the distal region 42 of the elongate shaft 40. The inflatable balloon 44 includes a deflated configuration and an inflated configuration. FIG. 3 shows the inflatable balloon 44 in its inflated configuration. The medical device 52 includes a tubular member 54 that extends relative to the elongate shaft 40 and along an outer surface 48 of the inflatable balloon 44. As will be discussed, the tubular member 54 includes one or more lumens that allows a conductive member forming an electrocautery electrode to be advanced and/or withdrawn within the tubular member 54.

In FIG. 3, the tubular member 54 extends interior to the elongate shaft 40 and exits the elongate shaft 40 through an aperture 56. The tubular member 54 extends along the outer surface 48 of the inflatable balloon 44 and includes a distal end 58 that is secured to a shaft segment 60 extending distally of the inflatable balloon 44. In some cases, the tubular member 54 may be adhesively secured to the outer surface 48 of the inflatable balloon 44. In some cases, the tubular member 54 may be secured relative to the inflatable balloon 44 at least in part by virtue of extending through the aperture 56. In some cases, the tubular member 54 may be secured relative to the inflatable balloon 44 at least in part by virtue of the distal end 58 of the tubular member 54 being adhesively secured to the shaft segment 60. In some cases, the shaft segment 60 may include a fitting that the distal end 58 of the tubular member 54 may extend through or be snap-fitted into.

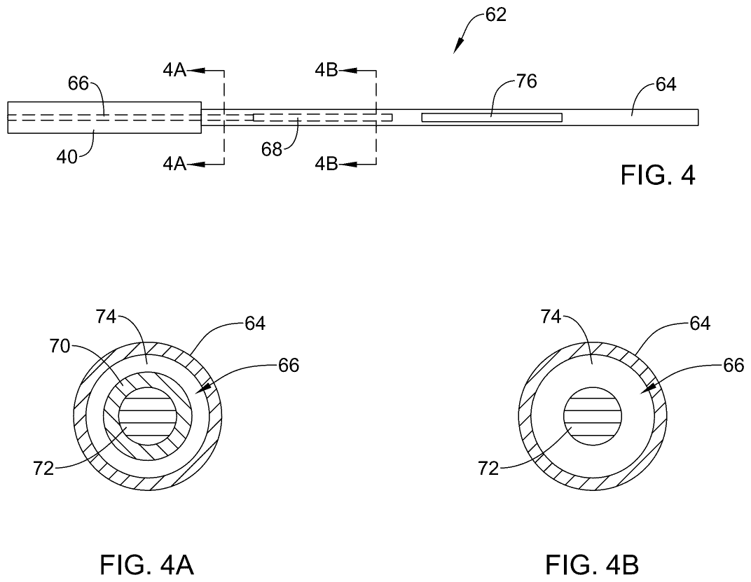

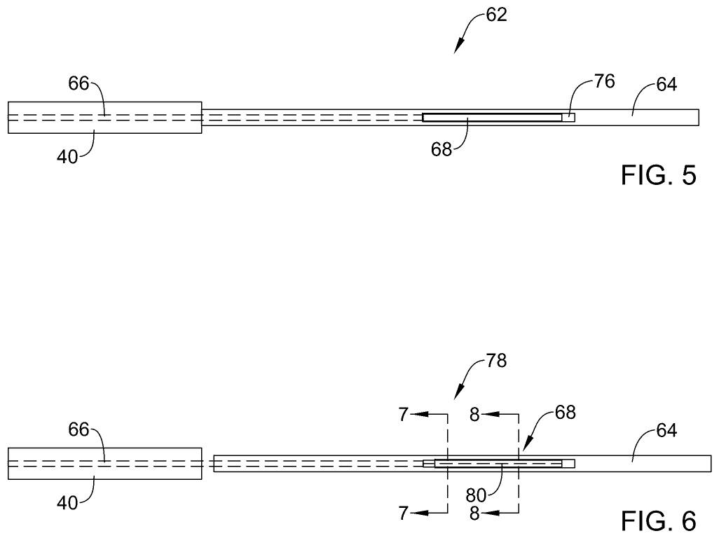

FIG. 4 is a schematic view showing an illustrative assembly 62. FIG. 5 is another schematic view of the illustrative assembly 62. The assembly 62 includes a tubular member 64 that extends out of the elongate shaft 40 and may be considered as representing either the tubular member 46 (FIG. 2) or the tubular member 54 (FIG. 3). A conductive member 66 extends within a lumen 74 (see FIGS. 4A and 4B) extending within the tubular member 64. In some cases, the conductive member 66 is insulated except for an electrocautery electrode 68 where an insulating layer extending over the conductive member 66 has been removed. This can be seen in FIGS. 4A and 4B, which are cross-sectional views taken along the lines 4A-4A and 4B-4B, respectively, of FIG. 4. In FIG. 4A, an insulative layer 70 covers a conductive core 72. In FIG. 4B, the insulative layer 70 has been removed, revealing the conductive core 72. Thus, the conductive core 72 forms the electrocautery electrode 68.

The tubular member 64 includes a window 76. In some cases, the window 76 may be an elongate slot that is formed within a wall of the tubular member 64. In some cases, the window 76 may be positioned within the tubular member 64 such that the window 76 faces away from an inflatable balloon (such as the inflatable balloon 44) to which the tubular member 64 is secured. Facing the window 76 away from the inflatable balloon 44 means that the electrocautery electrode 68 is better positioned to contact valve leaflet material. In some cases, the inflatable balloon 44 helps to urge the electrocautery electrode 68 into contact with valve leaflet material when the inflatable balloon 44 is inflated.

In FIG. 4, the electrocautery electrode 68 is not aligned with the window 76. Rather, the electrocautery electrode 68 is axially spaced from the window 76 and in this example, is located proximal of the window 76. The conductive member 66 may be moved distally within the lumen 74 such that the electrocautery electrode 68 is aligned with the window 76, as shown in FIG. 5, so that one of the valve leaflets 14 may be lacerated. In some cases, the electrocautery electrode 68 may be positioned within the lumen 74 at a location in which the electrocautery electrode 68 is distal of the window 76, and the conductive member 66 may be withdrawn proximally in order to align the electrocautery electrode 68 with the window 76 in order to lacerate one of the valve leaflets 14.

In some cases, the assembly 62 may be advanced into position near the valve leaflets 14 while the assembly 62 remains in the configuration shown in FIG. 4, i.e., with the electrocautery electrode 68 positioned either proximal of the window 76 or distal of the window 76. Positioning the assembly 62 may include translating the assembly 62. Positioning the assembly 62 may additionally or alternatively include rotating the assembly 62. After the assembly 62 has been positioned with the window 76 adjacent the valve leaflet 14 to be lacerated, the conductive member 66 may be translated within the tubular member 64 in order to position the electrocautery electrode 68 within the window 76. This may include advancing the conductive member 66 distally if the electrocautery electrode 68 was previously disposed proximal of the window 76. This may include retracting the conductive member 66 proximally if the electrocautery electrode 68 was previously disposed distal of the window 76. Once correctly positioned, with the electrocautery electrode 68 aligned with the window 76 and possibly extending radially outward of the window 76, an energy source may be activated to provide RF energy to the electrocautery electrode 68 and the valve leaflet 14 may be lacerated. Afterwards, the RF energy may be turned off and the assembly 62 may be withdrawn. In some cases, the assembly 62 may be repositioned in order to lacerate another valve leaflet 14.

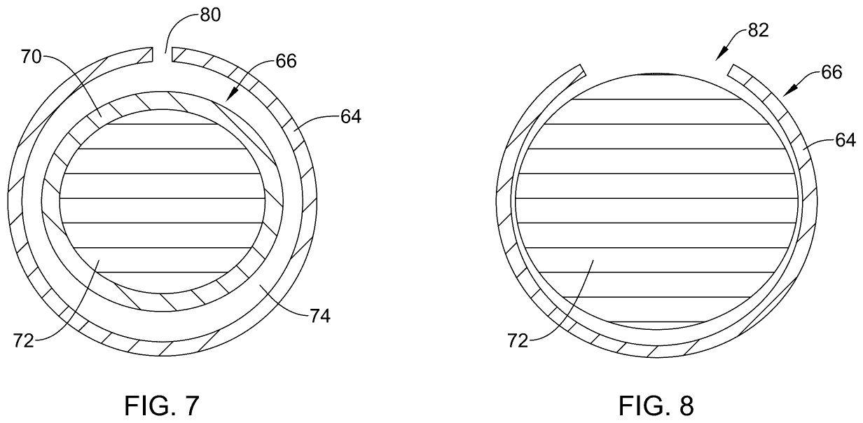

In some cases, the window 76 may not be an elongate slot formed within the wall of the tubular member 64, but may instead be a region of the tubular member 64 that is predisposed to split open. FIG. 6 is a schematic view of an illustrative assembly 78 in which the window 76 shown in FIGS. 4 and 5 has been replaced by a preferential tear line 80 that is formed within the tubular member 64. In some cases, part of the conductive member 66 may have a diameter that is greater than a diameter of the lumen 74. Extending the part of the conductive member 66 that has the increased diameter past the preferential tear line 80 may cause the preferential tear line 80 to split and expose a portion of the conductive member 66. As an example, the electrocautery electrode 68 may have a diameter that that is greater than a diameter of other parts of the conductive member 66 both distal and proximal of the electrocautery electrode 68. This may be demonstrated in FIGS. 7 and 8, which are cross-sectional views taken along the lines 7-7 and 8-8, respectively.

As seen in FIG. 7, which is a cross-sectional view through the conductive member 66 proximal of the electrocautery electrode 68, the conductive member 66, including the conductive core 72 and the accompanying insulation 70, has an outer diameter that is less than an inner diameter of the lumen 74. FIG. 8 is a cross-sectional view through the electrocautery electrode 68. As can be seen, the electrocautery electrode 68 has an outer diameter that is greater than the inner diameter of the lumen 74. This can stress the tubular member 64, which causes the tubular member 64 to split along the preferential tear line 80 (FIG. 6), forming a window 82. In some cases, the difference in diameter of the conductive member 66, between that shown in FIG. 7 and that shown in FIG. 8, is sufficient to open the preferential tear line 80 without damaging other parts of the tubular member 64. In some cases, tubular member 64 may split along a portion of or the entirety of the preferential tear line 80 (FIG. 6). In some cases, the difference in diameter of the conductive member 66, between that shown in FIG. 7 and that shown in FIG. 8, still permits the conductive member 66 to be translated within the tubular member 64.

In some cases, the assembly 78 may be advanced into position near the valve leaflets 14. Once a desired translational and rotational position is achieved, the conductive member 66 may be advanced through the tubular member 64 so that the enlarged diameter portion of the conductive member 66 passes the preferential tear line 80, causing the preferential tear line 80 to open and expose the electrocautery electrode 68 through an opening formed by splitting the preferential tear line 80. Once the electrocautery electrode 68 has been exposed, an energy source may be activated to provide RF energy to the electrocautery electrode 68 and the valve leaflet 14 may be lacerated. Afterwards, the RF energy may be turned off and the assembly 78 may be withdrawn. In some cases, the assembly 78 may be repositioned in order to lacerate another valve leaflet 14.

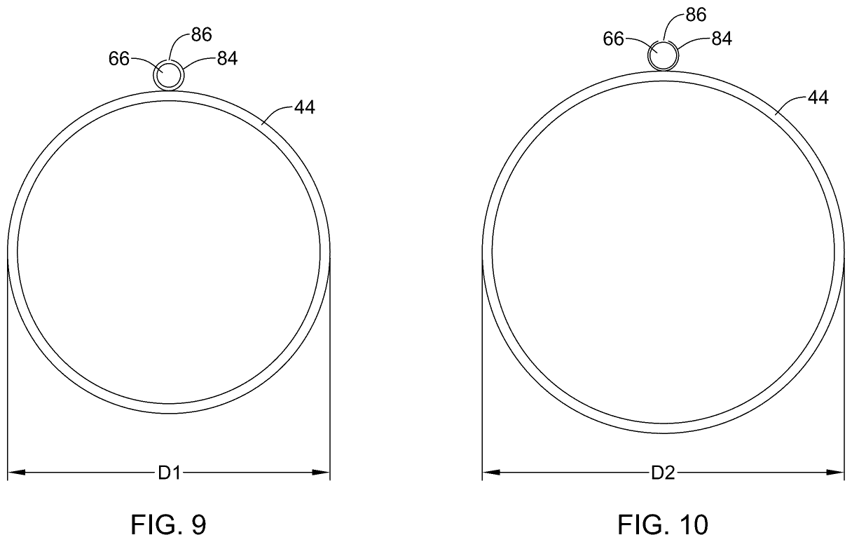

In some cases, rather than relying upon multiple diameters of the conductive member 66 to open a window to expose the electrocautery electrode 68, a window may be opened by varying the inflation pressure of the inflatable balloon 44. FIGS. 9 and 10 show a conductive core 66 disposed within a tubular member 84 that is secured to an outer surface of the inflatable balloon 44. In FIGS. 9 and 10, an assumption is made that the inflatable balloon 44 has been translated and/or rotated into a position in which the electrocautery electrode 68 is positioned to lacerate a valve leaflet 14 before the inflatable balloon 44 is inflated to a first pressure, as shown in FIG. 9.

The tubular member 84 may be considered as an example of the tubular members 46, 52, and 64. In FIG. 9, the inflatable balloon 44 has been inflated to a first pressure that is sufficient to give the inflatable balloon 44 a circular cross-sectional profile and an outer diameter labeled as D₁. The tubular member 84 includes a narrow slot 86 that is formed along a top surface of the tubular member 84. In FIG. 9, the narrow slot 86 is too narrow for the conductive core 66 to extend radially outwardly of the narrow slot 86.

In FIG. 10, the inflatable balloon 44 has been inflated to a second pressure that is greater than the first pressure, and that is sufficient to cause the inflatable balloon 44 to have a diameter labeled as D₂. As the inflatable balloon 44 increases in diameter as a result of the increased pressure within the inflatable balloon 44, the narrow slot 86 is caused to widen as the tubular member 84 is pushed outwardly by the increasing diameter of the inflatable balloon 44. The widening of the narrow slot 86 permits the conductive core 66 to be exposed, and to move radially away from the inflatable balloon 44. At this point an energy source may be activated to provide RF energy to the electrocautery electrode 68 and the valve leaflet 14 may be lacerated. Afterwards, the RF energy may be turned off and the inflatable balloon 44 may be deflated and withdrawn.

In some cases, the inflatable balloon 44 may be an at least partially compliant balloon in order to be able to control the overall diameter of the inflatable balloon 44 by controlling the inflation pressure of the inflatable balloon 44. In some cases, the diameter of the inflatable balloon 44 as shown in FIG. 9 may correspond to a pressure sufficient to inflate the inflatable balloon 44 without distending the inflatable balloon 44 while the diameter of the inflatable balloon 44 as shown in FIG. 10 may correspond to a pressure sufficient to distend the inflatable balloon 44.

In some cases, the inflatable balloon 44 as shown in FIG. 9 may be inflated to a pressure between 0 atm (atmospheres) and 1 atm (101.325 kilopascals (kPa)). In some cases, the inflatable balloon 44 as shown in FIG. 10 may be inflated to a pressure of about 6 atm (607.95 kPa). This difference in pressure may result in an overall diameter change of the inflatable balloon 44 (a difference between D₂ and D₁). In an example, the inflatable balloon 44 may have an overall diameter increase of 0.059 inches (1.5 millimeters) when changing from 1 atm to 6 atm. The inflatable balloon 44 may exhibit an overall diameter increase of 0.039 inches (1 millimeters) when changing from 1 atm to 4 atm (405.3 kPa). In some cases, the inflatable balloon 44 may have a hoop stress (at nominal pressure of 4 atm) of 1.87×10⁸ kPa (42,000 lb_{f}/mm²). The hoop stress may cause balloon distension, which causes the slot 86 to expand and expose the electrocautery electrode 68. In some cases, the inflatable balloon 44 may be a compliant balloon, which may result in larger diameter increases related to inflation pressure.

The materials that can be used for the various components of the devices and various elements thereof disclosed herein may include those commonly associated with medical devices. In some instances, the medical devices, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem) or Vestamid L21® available from Evonik Industries, elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof, or any other suitable material.

In at least some instances, portions or all of the medical devices described herein, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility is imparted into the medical devices and/or other elements disclosed herein. For example, the medical devices, and/or components or portions thereof, may be made of a material that does not substantially distort the image and create substantial artifacts (e.g., gaps in the image). Certain ferromagnetic materials, for example, may not be suitable because they may create artifacts in an MRI image. The medical assembly 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some instances, the medical devices and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative examples of the present disclosure, those of skill in the art will readily appreciate that yet other examples may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A medical device for lacerating valve leaflets, the medical device comprising:
an elongate shaft extending proximally from a distal region;
an inflatable balloon secured to the distal region, the inflatable balloon has an outer surface and includes a deflated configuration and an inflated configuration;
a tubular member extending relative to the elongate shaft and along the outer surface of the inflatable balloon, the tubular member includes a window faced away from the inflatable balloon; and
an electrocautery electrode slidingly disposed within the tubular member, the electrocautery electrode is translatable between a position in which the electrocautery electrode is axially spaced apart from the window and a position in which the electrocautery electrode is aligned with the window and is positioned to lacerate a valve leaflet.

2. The medical device of claim 1, wherein the electrocautery electrode comprises an uninsulated portion of an insulated conductive member slidingly disposed within the tubular member.

3. The medical device of claim 2, wherein the uninsulated portion of the insulated conductive member has a larger diameter than portions of the insulated conductive member on one or both sides of the uninsulated portion.

4. The medical device of claim 3, wherein the larger diameter uninsulated portion urges the window open as the larger diameter uninsulated portion approaches the window.

5. The medical device of claim 2, wherein the insulated conductive member is adapted to position the electrocautery electrode proximal of the window prior to translating the insulated conductive member distally in order to align the electrocautery electrode with the window.

6. The medical device of claim 2, wherein the insulated conductive member is adapted to position the electrocautery electrode distal of the window prior to translating the insulated conductive member proximally in order to align the electrocautery electrode with the window.

7. The medical device of claim 1, wherein the window comprises an opening in the tubular member.

8. The medical device of claim 1, wherein the window comprises an elongate slot formed in the tubular member.

9. The medical device of claim 8, wherein the tubular member is adapted such that inflating the inflatable balloon urges the elongate slot open.

10. The medical device of claim 1, wherein the window comprises a preferential tear region formed in the tubular member.

11. The medical device of claim 10, wherein the preferential tear region comprises a perforation formed in the tubular member.

12. The medical device of claim 1, wherein the valve leaflets comprise native valve leaflets.

13. A medical device for lacerating valve leaflets, the medical device comprising:
an elongate shaft extending proximally from a distal region;
an expandable element secured to the distal region, the expandable element movable between a compressed configuration and an expanded configuration;
a tubular member extending relative to the elongate shaft and exterior to the expandable element, the tubular member includes a side wall and an opening within the side wall; and
an electrode slidingly disposed within the tubular member, the electrode is translatable between a position in which the electrode is spaced apart from the opening and a position in which the electrode is aligned with the opening.

14. The medical device of claim 13, wherein the electrode comprises an uninsulated portion of an insulated conductive member slidingly disposed within the tubular member.

15. The medical device of claim 14, wherein the insulated conductive member is adapted to position the electrode proximal of the opening prior to translating the insulated conductive member distally in order to align the electrode with the window.

16. The medical device of claim 14, wherein the insulated conductive member is adapted to position the electrode distal of the opening prior to translating the insulated conductive member proximally in order to align the electrode with the window.

17. The medical device of claim 13, wherein the valve leaflets comprise native valve leaflets.

18. A medical device for lacerating valve leaflets, the medical device comprising:
an elongate shaft extending proximally from a distal region;
an inflatable balloon secured to the distal region, the inflatable balloon has an outer surface and includes a deflated configuration and an inflated configuration;
a tubular member extending relative to the elongate shaft and along the outer surface of the inflatable balloon, the tubular member includes a window faced away from the inflatable balloon; and
an insulated conductive member including an uninsulated portion thereof forming an electrode, the insulated conductive member slidingly disposed within the tubular member.

19. The medical device of claim 18, wherein the uninsulated portion of the insulated conductive member has a larger diameter than portions of the insulated conductive member on one or both sides of the uninsulated portion.

20. The medical device of claim 19, wherein the larger diameter uninsulated portion urges the window open as the larger diameter uninsulated portion approaches the window.
